(19) United States
(12) Patent Application Publication
(10) Pub. No.: US 2008/0208260 A1
(43) Pub. Date: Aug. 28, 2008

(54) SPINE TREATMENT DEVICES AND METHODS

(76) Inventors: Csaba Truckai, Saratoga, CA (US); John H. Shadduck, Tiburon, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/036,179

(22) Filed: Feb. 22, 2008

Related U.S. Application Data
(60) Provisional application No. 60/902,492, filed on Feb. 22, 2007.

(51) Int. Cl. A61B 17/58; A61F 2/44

(52) U.S. Cl. 606/280; 606/246; 623/17.11

(57) ABSTRACT

A modular implant system and method is provided for the dynamic stabilization of a spine segment and that can be implanted in a posterior approach. The implant system can include first and second support bodies configured for fixation to outward or lateral surfaces of first and second vertebrae, respectively. The implant system can also comprise a resilient portion. The method can comprise fixating first and second support bodies to first and second vertebrae respectively.

### CROSS-REFERENCE TO RELATED APPLICATIONS

This application claims the benefit of U.S. Provisional Application No. 60/902,492 filed Feb. 22, 2007, the entire contents of which are hereby incorporated by reference and should be considered a part of this specification. This application is also related to U.S. application Ser. No. 11/165,652, filed Jun. 24, 2005 which claims the benefit of Provisional U.S. Patent Application No. 60/633,509, filed Dec. 6, 2004. This application is also related to U.S. patent application Ser. No. 11/165,651, filed Jun. 24, 2005. The entire contents of all of the above applications are hereby incorporated by reference and should be considered a part of this specification.

### BACKGROUND OF THE INVENTION

1. Field of the Invention

The present invention relates generally to implant systems and methods for treating a spine disorder, and more particularly relates to braces and support members that can be configured for re-distributing loads within a spine segment while still allowing for flexion, extension, lateral bending and torsion. In certain embodiments, the system can include implants configured for fixation to lateral outward surfaces of two vertebrae.

2. Description of the Prior Art

Thoracic and lumbar spinal disorders and discogenic pain are major socio-economic concerns in the United States affecting over 70% of the population at some point in life. Low back pain is the most common musculoskeletal complaint requiring medical attention; it is the fifth most common reason for all physician visits. The annual prevalence of low back pain ranges from 15% to 45% and is the most common activity-limiting disorder in persons under the age of 45.

Degenerative changes in the intervertebral discs often play a role in the etiology of low back pain. Many surgical and non-surgical treatments exist for patients with degenerative disc disease (DDD), but often the outcome and efficacy of these treatments are uncertain. In current practice, when a patient has intractable back pain, the physician's first approach may be conservative treatment with the use of pain killing pharmacological agents, bed rest and limiting spinal segment motion. Only after an extended period of conservative treatment will the physician normally consider a surgical solution, that solution often being spinal fusion. Fusion procedures are highly invasive procedures that carry surgical risk as well as the risk of transition syndrome, wherein adjacent levels of the vertebrae will be at increased risk for facet and discogenic pain.

More than 150,000 lumbar and nearly 200,000 cervical spinal fusions are performed each year to treat common spinal conditions such as degenerative disc disease and spondylolisthesis, or misaligned vertebrae. Some 28 percent are multi-level, meaning that two or three vertebrae are fused. Such fusions “weld” unstable vertebrae together to eliminate pain caused by their movement. While there have been significant advances in spinal fusion devices and surgical techniques, the procedure does not always work reliably. In one survey, the average clinical success rate for pain reduction was about 75%; and long time intervals were required for healing and recuperation (3-24 months, average 15 months). Probably the most significant drawback of spinal fusion is termed the “transition syndrome” which describes the premature degeneration of discs at adjacent levels of the spine. This is certainly the most vexing problem facing relatively young patients when considering spinal fusion surgery.

Many spine experts consider the facet joints to be the most common source of spinal pain. Each vertebra possesses two sets of facet joints, one set for articulating to the vertebra above and one set for the articulation to the vertebra below. In association with the intervertebral discs, the facet joints allow for movement between the vertebrae of the spine. The facet joints are under a constant load from the weight of the body and are involved in guiding general motion and preventing extreme motions in the trunk. Repetitive or excessive trunkal motions, especially in rotation or extension, can irritate and injure facet joints or their encasing fibers. Also, abnormal spinal biomechanics and bad posture can significantly increase stresses and thus accelerate wear and tear on the facet joints.

Recently, technologies have been proposed or developed for disc replacement that may replace, in part, the role of spinal fusion. The principal advantage proposed by complete artificial discs is that vertebral motion segments can retain some degree of motion at the disc space that otherwise would be immobilized in more conventional spinal fusion techniques. Artificial facet joints are also being developed. Many of these technologies are in clinical trials. However, such disc replacement procedures are still highly invasive procedures which require an anterior surgical approach through the abdomen.

Clinical stability in the spine can be defined as the ability of the spine under physiologic loads to limit patterns of displacement so as to not damage or irritate the spinal cord or nerve roots. In addition, such clinical stability can prevent incapacitating deformities or pain due to later spine structural changes. Any disruption of the components that stabilize a vertebral segment (i.e., discs, facets, ligaments) decrease the clinical stability of the spine.

Improved devices and methods are needed for treating dysfunctional intervertebral discs and facet joints to provide clinical stability. There is a particular need for: (i) implantable devices that can offset vertebral loading to treat disc degenerative disease and facets, such devices being introduced through the least invasive procedures; (ii) implants and systems that can restore disc height and foraminal spacing; and (iii) implants and systems that can re-distribute loads in spine flexion, extension, lateral bending and torsion.

### SUMMARY OF THE INVENTION

In accordance with one embodiment, an implant system for treating an abnormal spine segment of a patient is provided. The implant system can comprise a first plate configured for fixation to a lateral surface of a vertebral body of a first vertebra, a second plate configured for fixation to a lateral surface of a vertebral body of a second vertebra and an intermediate body. The intermediate body can be disposed between the first and second plates along the lateral surfaces of the first and second vertebrae.

In an additional embodiment a method of extradiscal support of an abnormal spine segment of a patient is provided. The method may comprise the steps of fixating a first plate on a lateral surface of a vertebral body of a first vertebra and fixating a cooperating second plate on a lateral surface of a vertebral body of an adjacent vertebra. A resilient intermediate body portion may be positioned between the first and second plates, the intermediate body portion disposed between opposing surfaces of the first and second plates to thereby alter load-carrying characteristics of the spine segment. The resilient intermediate body portion may also allow for controlled axial, bending and rotational movement of the first plate relative to the second plate and relative to the axis of the spine.

A further embodiment provides a brace for treating an abnormal bone joint in a human patient. The brace can comprise first and second plates configured for fixation to lateral outer surfaces of first and second bones spaced apart by a joint, each of the plates having contoured surface configured to substantially conform to the lateral outer surfaces of the first and second bones. An intermediate support structure of a resilient material is intermediately coupled to the first and second plates, the intermediate support structure configured to allow for the controlled axial, bending and rotational movement of the first bone relative to the second bone. The support structures may off-load the joint.

In accordance with another embodiment, an extradiscal support system for treating an abnormal spine segment is provided. The system can comprise first and second support members configured for transpedicular introduction and fixation to lateral surfaces of vertebral bodies of first and second vertebra. The system may further comprise an extension member coupleable to the first and second support members.

An additional embodiment may comprise a method of extradiscal support of an abnormal spine segment of a patient. The method may comprise the steps of fixating paired support members extending outward from lateral surfaces of first and second vertebral bodies of first and second vertebrae, the support members introduced transpedicularly through the first and second vertebral bodies, and introducing an extension member between head portions of the paired support members to thereby off-load a disc.

Other implant systems and methods within the spirit and scope of the invention can be used to increase intervertebral spacing, increase the volume of the spinal canal and off-load the facet joints to thereby reduce compression on nerves and vessels to alleviate pain associated therewith.

### BRIEF DESCRIPTION OF THE DRAWINGS

The features and advantages of this invention, and the manner of attaining them, will become apparent by reference to the following description of preferred embodiments of the invention taken in conjunction with the accompanying drawings, wherein:

FIG. 1 is a schematic side view of a patient's spine segment with one embodiment of an implant system.

FIG. 2 is a schematic posterior view of the implant system of FIG. 1

FIG. 3 is a schematic sectional view of the implant device of FIG. 1.

FIG. 4 is a schematic side view of an alternative embodiment of a spine implant system.

FIG. 5 is a schematic side view of an alternative embodiment of a spine implant system.

FIG. 6 is a schematic side view of an alternative embodiment of a spine implant system.

FIG. 7A is a schematic sectional view of a step of one embodiment of a transpedicular method involving an alternative spine implant system.

FIG. 7B is a sectional view of an alternative support member introduced through a transpedicular method.

FIG. 7C is a side view of an alternative embodiment of a spine implant system.

### DETAILED DESCRIPTION OF PREFERRED EMBODIMENTS

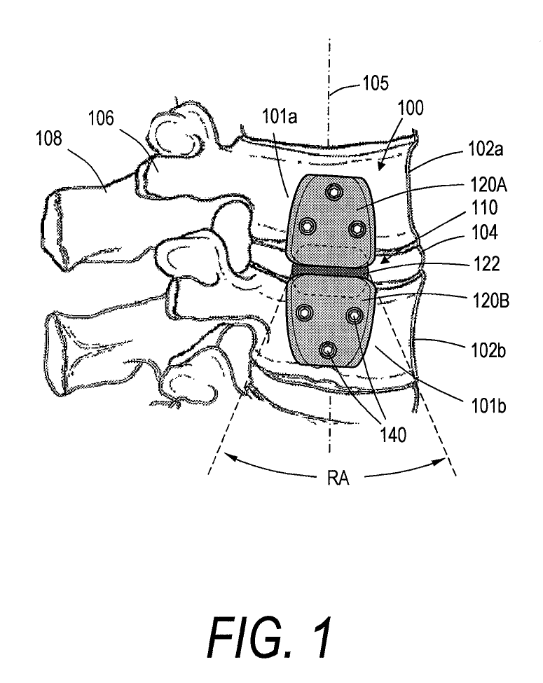

FIGS. 1-3 show a spine segment and one embodiment of a spine implant system, the implant system having an implant body or assembly 100 extending between outward or lateral surfaces 101a and 101b of first and second vertebra 102a and 102b. It can be seen that the intermediate portion 110 of the implant body 100 spans disc 104, with the implant body generally extending in a superior-inferior direction in alignment with axis 105 of the spine segment. The implant body 100 can extend between lateral or outward surfaces of the anterior vertebra, as opposed to the pedicles 106 or spinous processes 108. In a preferred embodiment the first and second vertebrae 102a and 102b can be lumbar or thoracic vertebrae. In another embodiment the first and second vertebra 102a and 102b can be cervical vertebrae.

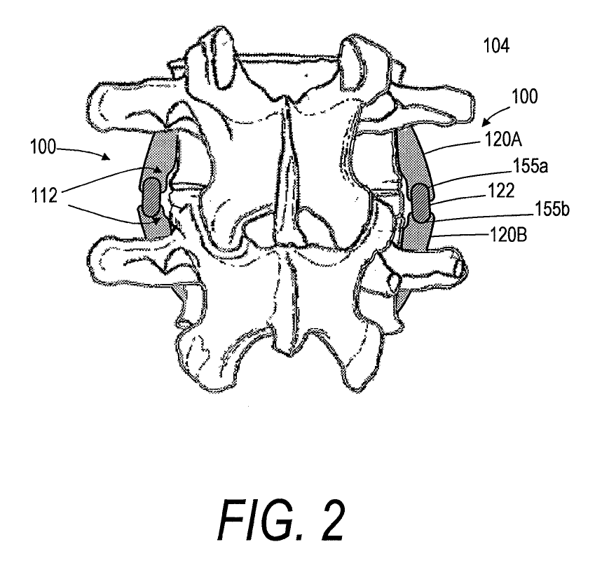

Still referring to FIGS. 1 and 2, each implant body 100 can have a first (e.g., superior) body portion 120A and a second (e.g., inferior) body portion 120B. In some embodiments, as in FIG. 1, the implant can include an intermediate body portion 122 disposed between the first and second body portions 120A, 120B. In the illustrated embodiments the first and second body portions 120A, 120B can be plates 120A, 120B. The plates 120A, 120B can preferably be contoured to substantially conform to the outer or lateral surfaces 101a, 101b of the vertebral bodies of the vertebrae 102a, 102b. In addition, as can be seen in FIG. 2, the plates 120A, 120B can have a recess 112 on its inner surface near the junction with the intermediate body portion 122 to generally accommodate the geometry of the vertebral bodies proximate the intervertebral disc 104. In other embodiments the plates may be flat.

In one embodiment, at least part of the intermediate body portion 122 can be made of a resilient material. In a preferred embodiment the entire intermediate body portion 122 can be made of a resilient material. For example, the intermediate body portion 122 may be a resilient block or rod. In some embodiments, the intermediate body portion 122 can comprise at least in part an elastomer. For example, the resilient material of the intermediate body portion 122 can be selected from the group of Silicone rubber, Urethane, Polybutadiene (BR), Styrene-butadiene rubber (SBR), THERBAN®, ZETPOL®, VITON®, TECNOFLON®, FLUOREL®, DAI-EL®, HYPANLON®, KALREZ®, HYTREL®, fluoroelastomers (FKM, FPM) and SANTOPRENE®.

Each of the first and second body portions 120A and 120B can be fixated to the bone surface with a fixation mechanism, which in the illustrated embodiment includes at least one anchor 140. In one embodiment, the anchor 140 can be a bone screw. However, the anchor can include any suitable fastening mechanism. In the illustrated embodiment, each of the first and second body portions 120A, 120B has three anchors 140. However, one of ordinary skill in the art will recognize that any fewer or more anchors can be used to fasten the first and second body portions 120A, 120B to the respective vertebrae 102a, 102b. The first and second body portions 120A and 120B are preferably fixed or anchored to the respective vertebrae 102a, 102b so as to avoid interaction with any of the nerves or blood vessels proximate the vertebrae 102a, 102b. For example, the first and second body portions 120A, 120B, can be fixed to the lateral surfaces 101a, 101b of the vertebrae 102a, 102b at a location anterior of the spinal cord and other nerves.

The first and second body portions 120A and 120B can be fabricated of any biocompatible material, such as metal or plastic, and may, in one embodiment, have a gripping surface (e.g., spiked, rough, rasp-like) for engaging the bone surface, for example, when a single anchor 140 is used. The fixation mechanism can in one embodiment also include the use of bone cement.

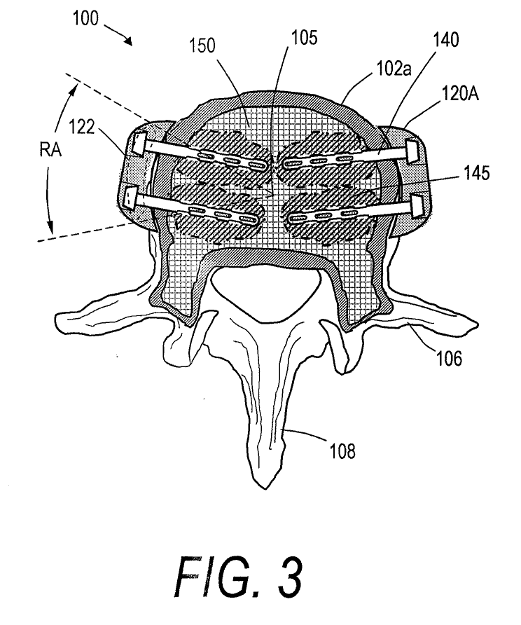

As can be seen in FIG. 3, one fixation mechanism or anchor can be found in co-pending U.S. application Ser. No. 11/780,967 filed Jul. 20, 2007, which is incorporated herein by reference in its entirety, and wherein an elongated anchor 140 can have a bore extending therethrough and through which bone cement 145 can be injected into cancellous bone 150 of a vertebral body 102a, 102b. In some embodiments, a bone cement source can be coupled to the anchor 140 to deliver bone cement through the bore in the anchor 140 and into cancellous bone 150 of the vertebra 102a. In some embodiments, the anchor can include a heating element or thermal emitter for altering the viscosity of the bone cement as disclosed, among other things, in several pending applications listed above in the section titled CROSS-REFERENCE TO RELATED APPLICATIONS. In some embodiments the anchor can have a flex-portion.

It can be seen from FIGS. 1 and 2, that the paired implant bodies 100 on opposing sides of the vertebrae can off-load the disc 104. In some embodiments, the first support body 120A and second support body 120B can be fixed to lateral surfaces of adjacent vertebra. The first and second support bodies 120A, 120B may also have surface features that allow for retention of the intermediate resilient body 122 between opposing surfaces 155a and 155b of the first and second support bodies 120A, 120B. For example, in some embodiments, the intermediate resilient body 122 may be captured or tethered between the first and second support bodies 120A, 120B. In other embodiments, the surfaces 155a and 155b for retaining the intermediate resilient body portion 122 can have at least one feature selected from the group consisting of a concavity, a recess, a groove, a cut, an indentation, an undulation, a slot and a bore. Further, the intermediate resilient body portion 122 can have a cooperating or coupling feature that is received by said surface feature of the support body 120A, 120B. The coupling feature can be selected from the group of a snap-fit, a tether, a cord, and cooperating projecting element that is coupled to a said surface feature of the support body 120A, 120B. In another embodiment, the intermediate resilient body portion 122 is bonded to either or both support bodies. However, other suitable mechanisms for retaining the intermediate body portion 122 in between the support body portions 120A, 120B can be used.

It can be understood that the implant system and intermediate resilient body portion 122 can allow movement of the first support body 120A relative to the second support body 120B around the intermediate resilient body portion 122, for example axially relative to the axis 105 of the spine segment. For example, the resilient portion 122 can bend or twist to allow a change in the orientation, or torsion, of the first support body 120A relative to the second support body 120B. Also, the implant system and intermediate resilient body 122 can allow movement of the first support body 120A relative to the second support body 120B rotationally relative to the axis 105 of the spine segment. In some embodiments, the implant system and resilient body portion 122 can allow or disallow movement of the first support body 120A relative to the second support body 120B transverse to the axis 105 of the spine segment, depending on surface features of the support bodies 120A, 120B.

Thus, in one embodiment, a method for treating an abnormal spine segment can include fixating a first support body 120A on a lateral surface of a first vertebra 102a and fixating a cooperating second support member 120B on an adjacent lateral surface of an adjacent vertebra 102b, wherein opposing surfaces 155a, 155b of the first and second support members include retaining features for retaining the resilient body portion 122 therebetween, which thereby alters load-carrying characteristics of the treated spine segment. The method can further include providing a resilient body portion 122 that allows for controlled axial, bending and rotational movement of the first support body relative to the second support body and relative to the axis 105 of the spine. Further, the method can include substantially disallowing or inhibiting transverse movement of the first support body 120A relative to the second support body 120B and relative to the axis 105 of the spine.

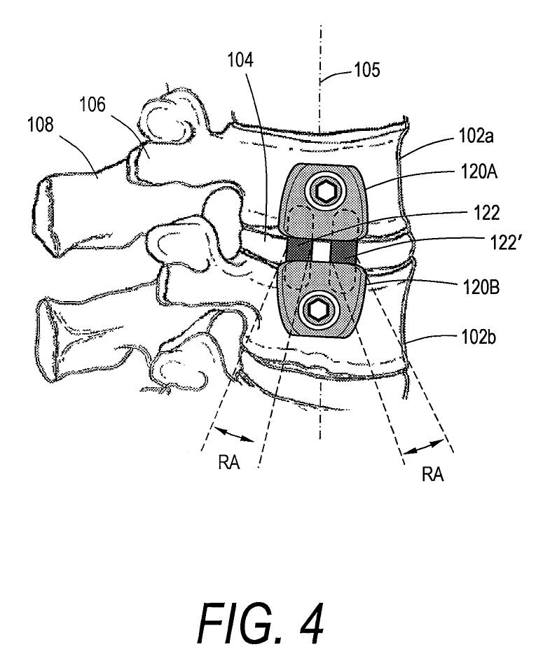

Referring to FIGS. 1 and 4, some embodiments of a treatment assembly can be described as an extradiscal support system for treating an abnormal spine segment of a human patient, comprising paired first and second support structures 120A and 120B which can be fixed to at least one lateral outward surface 101a, 101b of first and second vertebrae 102a, 102b, and an intermediate member which can include a resilient portion 122. The resilient portion 122 can engage opposing surfaces of the paired first and second support structures 120A, 120B, and the width of the resilient portion 122 can extend a radial angle RA of at least 10° relative to the axis 105 of the spine segment (see FIG. 3). For example, the radial angle RA can span a segment (e.g., an arc) that defines the width of the resilient portion 122.

In other embodiments of an extradiscal support system, the cross-section of the resilient portion 122 can extend a radial angle RA of at least 15°, 20°, 25°and 30° relative to the axis 105 of the spine segment. In some embodiments the resilient portion 122 can have a width of between about 5 mm and 15 mm. In some embodiments the width may be about 20 mm. The resilient portion 122 can have a curved shape. The curved surface may have an arc length of between about 5 mm and 15 mm that is defined by the radial angle RA. In some embodiments the arc length may be about 20 mm. In some embodiments the resilient portion 122 may be shaped like a section of a donut. This design can be advantageous in that the resilient portion 122 can act as an outward support or extension of a diseased disc 104.

The dimensions of the resilient portion 122 can vary based on the embodiment. For example, the resilient portion 122 in one embodiment may have a height or length of at least 1 mm. In a preferred embodiment the resilient portion 122 can be between about 2 mm to 10 mm high. In other embodiments the resilient portion 122 can be taller than this. In some embodiments the resilient portion 122 can have a thickness of between about 2 to 6 mm. Other embodiments are not limited to any of the above disclosed dimensions.

In some embodiments, the first and second body portions 120A, 120B may be between about 5 mm to 20 mm wide.

In one embodiment, as shown in FIG. 1, the resilient portion 122 can be a single member. In another embodiment, shown in FIG. 4, the resilient portion can comprise a plurality of members 122, 122′, wherein the number can range from 2 to about 100 members or elements. In another embodiment, the resilient elements, such as the resilient portion 122 in FIG. 1 or resilient members 122, 122′ in FIG. 4, can also be fabricated of shape memory alloys (NiTi), helical spring elements, flat spring elements and the like.

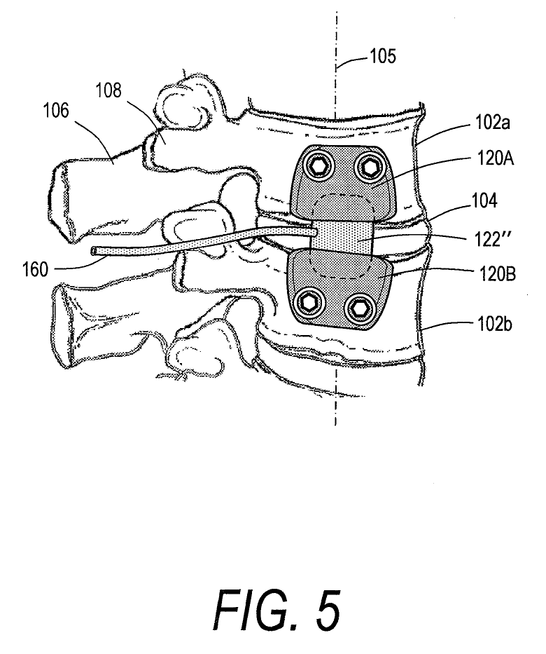

In other embodiments of an extradiscal support system, such as in FIG. 5, the cross-section of the resilient portion 122″ can have adjustable parameters selected from the group consisting of transverse dimension, vertical dimension, radial dimension and modulus. In the illustrated embodiment, the resilient portion can be expandable in whole or in part. The resilient portion can have an interior region or chamber that is expandable (e.g., via the introduction of a fluid therein). In FIG. 5, the resilient portion 122″ can be expanded with an inflation tube 160 coupled to the resilient portion 122″. In other embodiments, the extradiscal support system can comprise an electrical connection mechanism within resilient portion 122″ for the purpose of coupling an energy source to the resilient portion of a shape memory polymer which can adjust the polymer from a temporary shape to or toward a memory shape. The electrical connection mechanism may comprise, for example, wires, leads, or electrical contacts. By adjusting the parameters discussed above, the extradiscal support system may be able to adjust the off-loading of at-least one disc 104.

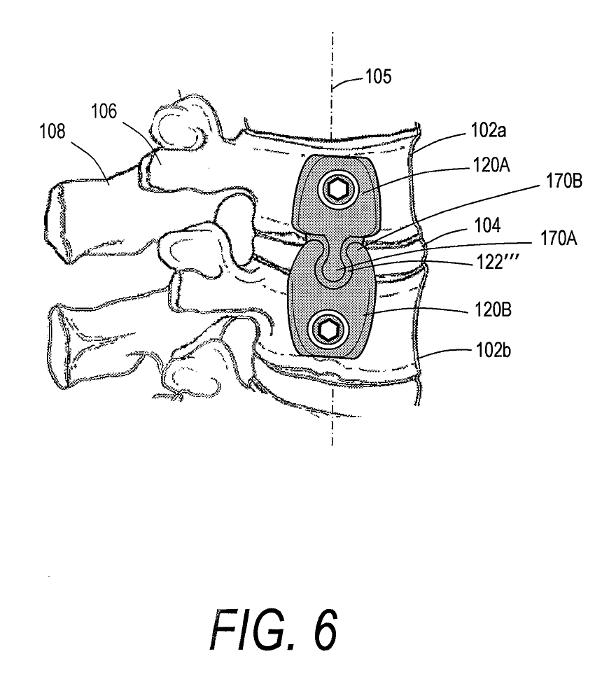

Referring to FIG. 6, in other embodiments of an extradiscal support system, the system or brace can include first and second support structures 120A and 120B which can be fixated to outer surfaces of first and second bones spaced apart by a joint, and an intermediate support structure of a resilient material intermediately coupled to the first and second support structures 120A, 120B, with cooperating “key” shapes 170A, 170B. Intermediate the key shapes or features indicated at 170A and 170B can be a resilient material 122′″ for flexibly off-loading at least one disc 104. Such a brace can typically be configured for fixation to adjacent vertebrae, but also can be configured for fixation to non-adjacent vertebrae to off-load at least two discs.

Any implant body as depicted in the Figures above also can be fabricated with the extension elements or resilient member(s) 122 that include a shock-absorber or spring-form such as a helical form, arcuate form or any compound curvature for functioning as a spring.

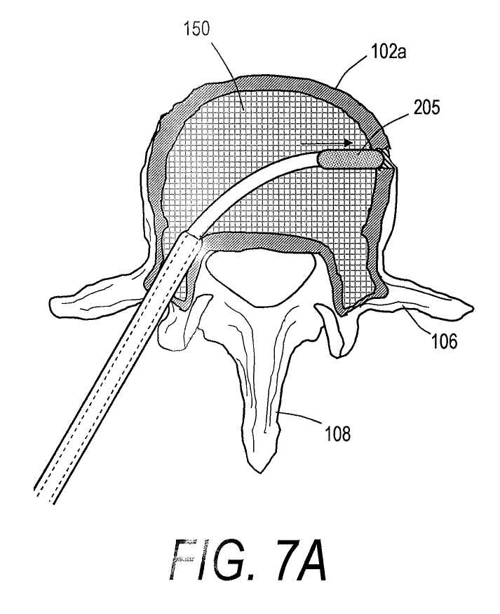

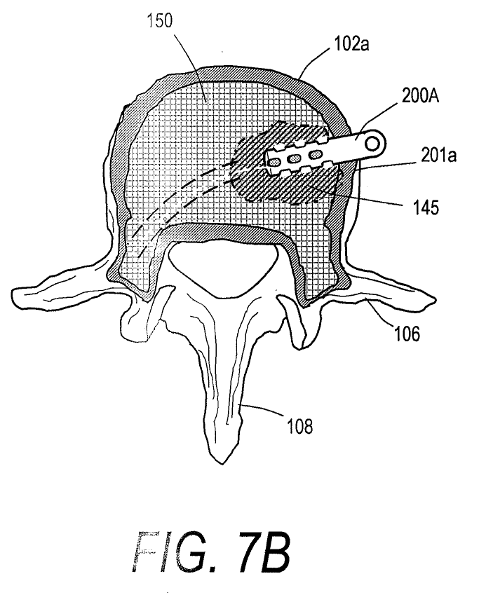

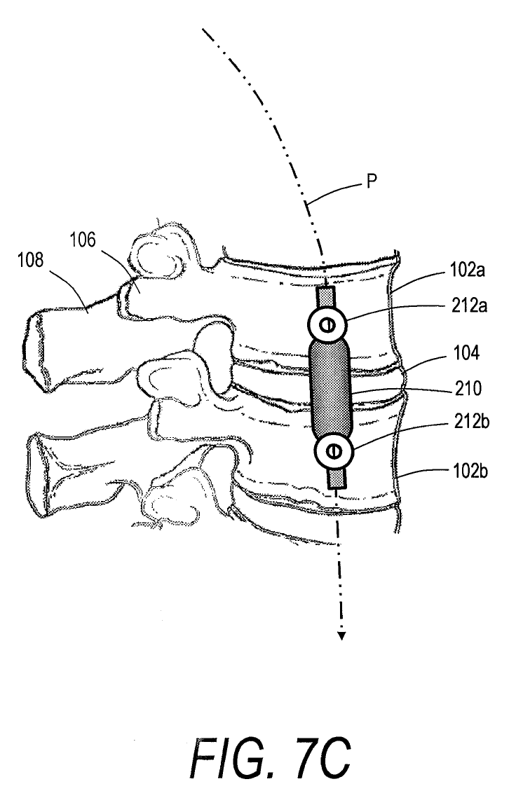

The embodiments above illustrate implant bodies that can utilize fixation mechanisms introduced from the exterior of the lateral vertebral body surface. In another embodiment, a transpedicular approach is possible in adjacent vertebrae (or spaced apart vertebrae) wherein fixated support members can be introduced from inside the vertebra to the outside of the lateral surfaces. Referring now to FIG. 7A, a directional drill tip 205 can be introduced transipedicularly to prepare the vertebra 102a for the insertion of a support member, such as support member 200A in FIG. 7B. FIG. 7B shows the hardening of bone cement 145 after the support member 200A has been put in place from inside the vertebra 102a. A part of the support member 200A may be outside of the lateral surface 201a of the vertebra. FIG. 7C shows a path P along which an extension member 210 may be introduced. FIG. 7C also shows the extension member 210 connected to the support members after receiving apertures of head portions 212a and 212b of the support members. This step can be accomplished in a minimally invasive manner using a first guidewire followed by an extension member over the guidewire in some embodiments.

As shown in FIGS. 7A-7C, which may also make up part of other embodiments, the extension member 210 can be an expandable rod with hardenable fill material, or a thermally hardenable rod as described, among other things, in co-pending U.S. application Ser. No. 11/780,967, referenced previously. In some embodiments, the extension member 210 can include at least one resilient body portion. In other embodiments, the extension member 210 can include a shock absorber. In still additional embodiments, the extension member 210 can extend a radial angle of at least 10° relative to the axis of the spine segment including a shock absorber.

The implant systems described in at least one embodiment above provide implants and treatment methods that can off-load a spine segment with extradiscal support structures provided on lateral outward surfaces of adjacent vertebrae. Advantageously, the implant body 100 described above off-loads a diseased intervertebral disc by providing a resilient portion, such as the resilient portion 122, which functions as a supplemental disc and is attached to support members 120A, 120B attached to the vertebral bodies on either side of the diseased disc.

In certain embodiments, the implant system can be considered for use by physicians far in advance of more invasive fusion or disc replacement procedures. In certain embodiments, the implant system allows for dynamic stabilization of a spine segment in a manner that is comparable to complete disc replacement. Embodiments of the implant system are configured to improve on disc replacement procedures in that it can augment vertebral spacing (e.g., disc height) and foraminal spacing at the same time as controllably reducing loads on facet joints—which complete disc replacement may not address. Certain embodiments of the implant system is based on principles of a native spine segment by creating stability with a tripod load receiving arrangement. The implant arrangement thus supplements the spine's natural tripod load-bearing system (disc and two facet joints) and can re-distribute loads with the spine segment in spine torsion, extension, lateral bending and flexion.

Of particular interest, since the embodiments of implant systems are far less invasive that artificial discs and the like, the systems likely may allow for a rapid regulatory approval path when compared to the more invasive artificial disc procedures.

Although these inventions have been disclosed in the context of a certain preferred embodiments and examples, it will be understood by those skilled in the art that the present inventions extend beyond the specifically disclosed embodiments to other alternative embodiments and/or uses of the inventions and obvious modifications and equivalents thereof. For example, any of the implants disclosed above can be made of a metal material, polymer material, a shape memory alloy, or any suitable material for use in spinal implants. In addition, while a number of variations of the inventions have been shown and described in detail, other modifications, which are within the scope of the inventions, will be readily apparent to those of skill in the art based upon this disclosure. It is also contemplated that various combinations or subcombinations of the specific features and aspects of the embodiments may be made and still fall within one or more of the inventions. Accordingly, it should be understood that various features and aspects of the disclosed embodiments can be combine with or substituted for one another in order to form varying modes of the disclosed inventions. Thus, it is intended that the scope of the present inventions herein disclosed should not be limited by the particular disclosed embodiments described above. Although particular embodiments of the present invention have been described above in detail, it will be understood that this description is merely for purposes of illustration. Specific features of the invention are shown in some drawings and not in others, and this is for convenience only and any feature may be combined with another in accordance with the invention. Further variations will be apparent to one skilled in the art in light of this disclosure and are intended to fall within the scope of the appended claims.

1. An implant system for treating an abnormal spine segment of a patient, the spine segment extending about an axis, comprising:
a first plate configured for fixation to a lateral surface of a vertebral body of a first vertebra;
a second plate configured for fixation to a lateral surface of a vertebral body of a second vertebra; and
an intermediate body disposed between the first and second plates along the lateral surfaces of the first and second vertebrae.

2. The implant system of claim 1, wherein the intermediate body comprises a resilient portion.

3. The implant system of claim 2, wherein the resilient portion comprises at least in part an elastomer.

4. The implant system of claim 2, wherein the resilient portion is selected from the group consisting of Silicone rubber, Urethane, Polybutadiene (BR), Styrene-butadiene rubber (SBR), THERBAN®, ZETPOL®, VITON®, TECNOFLON®, FLUOREL®, DAI-EL®, HYPALON®, KALREZ®, HYTREL®, fluoroelastomers (FKM, FPM) and SANTOPRENE®.

5. The implant system of claim 2, wherein the intermediate body is retained between the first and second plates by a surface feature in at least one of the plates, the surface feature selected from the group consisting of a concavity, a recess, a groove, a cut, an indentation, an undulation, a slot and a bore.

6. The implant system of claim 5, wherein the intermediate body has a cooperating feature that is received by said surface feature of the at least one of the plates.

7. The implant system of claim 1, wherein the intermediate body is bonded to a plate.

8. The implant system of claim 1, wherein the intermediate body is configured to allow movement of the first plate relative to the second plate axially and rotationally relative to the axis of the spine segment.

9. The implant system of claim 1, wherein the intermediate body is configured to inhibit movement of the first plate relative to the second plate transverse to the axis of the spine segment.

10. The implant system of claim 1, wherein the width of the intermediate body extends a radial angle of at least 10° relative to the axis of the spine segment.

11. The implant system of claim 1, wherein the width of the intermediate body extends a radial angle of at least 30°.

12. The implant system of claim 1, wherein the plates are between 5 and 20 mm wide.

13. The implant system of claim 2 wherein the resilient portion has adjustable parameters selected from the group consisting of transverse dimension, vertical dimension, radial dimension and modulus.

14. The implant system of claim 13, wherein the resilient portion is expandable.

15. The implant system of claim 14, further comprising an inflation tube coupled to the resilient portion.

16. The implant system of claim 1, wherein the intermediate portion comprises a plurality of resilient elements.

17. The implant system of claim 2, wherein the resilient portion is a shape memory polymer.

18. A method of extradiscal support of an abnormal spine segment of a patient, comprising:
fixating a first plate on a lateral surface of a vertebral body of a first vertebra;
fixating a cooperating second plate on a lateral surface of a vertebral body of an adjacent second vertebra; and
positioning a resilient intermediate body portion between the first and second plates, the intermediate body portion disposed between opposing surfaces of the first and second plates to thereby alter load-carrying characteristics of the spine segment,
wherein the resilient intermediate body portion allows for controlled axial, bending and rotational movement of the first plate relative to the second plate and relative to an axis of the spine segment.

19. The method of claim 18, further comprising the steps of:
fixating a third plate on an opposing lateral surface of the vertebral body of the first vertebra body, the third plate positioned on an opposite side of the spine segment from the first plate; and
fixating a fourth plate on an opposing lateral surface of the vertebral body of the second vertebral body, the fourth plate positioned on an opposite side of the spine segment from the second plate.

20. The method of claim 18, wherein the intermediate resilient body portion inhibits transverse movement of the first plate relative to the second plate and relative to the axis of the spine.

21. The method of claim 18, wherein fixating includes inserting at least one anchor member to fix the plates to the vertebrae at locations that inhibit interaction with nerves and blood vessels proximate the vertebrae.

22. The method of claim 18, further comprising retaining the intermediate body portion between the first and second plates.

23. The method of claim 22, wherein retaining comprises bonding the resilient intermediate body portion and at least one plate.

24. The method of claim 18, wherein fixating includes deployment of at least one bone screw at least partly fixated in the vertebra with a bone cement.

25. The method of claim 18, further comprising adjusting a functional parameter of the resilient intermediate body portion chosen from the group consisting of a transverse dimension, a vertical dimension, a radial dimension and a modulus of the resilient intermediate body portion.

26. The method of claim 21, wherein inserting at least one anchor member is anterior of nerves within the vertebral body.

27-40. (canceled)
